(19) United States
(12) Plant Patent Application Publication
(10) Pub. No.: US 2016/0242339 P1
(43) Pub. Date: Aug. 18, 2016

(54) Grapevine named 'SAUVIGNON DORÉ'

(50) ;

(71) Applicants: Università degli Studi di Udine, Udine UD (IT); Istituto di Genomica Applicata, Udine UD (IT)

(72) Inventors: Simone Diego Castellarin, Udine (IT); Guido Cipriani, Faedis (IT); Gabriele Di Gaspero, Cividale del Friuli UD (IT); Michele Morgante, Tricesimo UD (IT); Enrico Peterlunger, Codroipo UD (IT); Raffaele Testolin, Udine (IT)

(73) Assignees: Università degli Studi di Udine, Udine UD (IT); Istituto di Genomica Applicata, Udine UD (IT)

(21) Appl. No.: 14/545,100

(22) Filed: Mar. 26, 2015

(30) Foreign Application Priority Data
Feb. 13, 2015 (QZ) 2015/0359

(51) Int. Cl. A01H 5/00

(52) U.S. Cl. PLT/207

(57) ABSTRACT

A new and distinct variety of grapevine named ‘SAUVIGNON DORÉ’, primarily adapted to the growing conditions of the temperate regions, and characterized by its medium vigor; semi-erect growth habit; wedge-shaped leaves with medium texture; small cylindrical shaped green-yellow berry clusters with a golden nuance on the sunny side of the berries; early maturing, soft berry flesh with no coloration and neutral taste primarily for winemaking; and resistance to winter temperatures (to −23° C.), resistance to downy mildew and tolerance to powdery mildew.

### LATIN NAME OF THE GENUS AND SPECIES OF THE PLANT CLAIMED

Vitis×vinifera (hybrid)

### VARIETY NAME

‘SAUVIGNON DORÉ’

### BACKGROUND OF THE INVENTION

The present invention relates to a new and distinct summer/fall bearing grapevine variety, botanically known as Vitis vinifera, and hereinafter referred to by the name ‘SAUVIGNON DORÉ’.

The new grapevine ‘SAUVIGNON DORÉ’ is a product of a controlled breeding program conducted by the inventors in Udine, Italy. The objective of the breeding program was to develop a new grapevine variety particularly characterized by resistance to cold (<−20° C.), resistance to downy mildew (Plasmopara viticola), and tolerance to powdery mildew (Uncinula necator).

The new grapevine ‘SAUVIGNON DORÉ’ originated from a cross made by the inventors in 2002 in Udine, Italy. The female or seed parent is the grapevine variety Vitis vinifera cv. ‘Sauvignon’ (unpatented), and the male or pollen parent is the grapevine variety Vitis cv. ‘Bianca’ (Eger 2×Bouvier) (unpatented).

The new grapevine ‘SAUVIGNON DORÉ’ was discovered and selected by the inventors as a single flowering plant within the progeny of the stated cross in a controlled environment in 2002 in Udine, Italy. Asexual reproduction of the new grapevine variety by grafting was first performed in February 2004 in Rauscedo, Friuli Venezia Giulia region, Italy, and has demonstrated that the combination of characteristics as herein disclosed for the new cultivar are firmly fixed and retained through successive generations of asexual reproduction. The new cultivar reproduces true to type.

### SUMMARY OF THE INVENTION

‘SAUVIGNON DORÉ’ is primarily adapted to the climate and growing conditions of the temperate regions with average yearly temperature about 13° C., minimum winter temperature about −20° C., annual rainfall around 700-1500 mm of rain (e.g. North-Eastern Italy, Friuli). This region provides the necessary year-round temperatures required to produce and maintain a strong vigorous plant with consistent fruit production from April through November on primocanes and in the ensuing year from April through November on the floricanes.

The following traits have been repeatedly observed and are determined to be unique characteristics of ‘SAUVIGNON DORÉ’, which in combination distinguish this grapevine plant as a new and distinct variety.
1. Medium vigor;
2. Semi-erect growth habit;
3. Medium size wedge-shaped leaves, with a pale green upper and lower surface and medium texture;
4. Small, dense cylindrical clusters with 1 small size wing; early maturing, green-yellow berries with golden nuances on the sunny side of the berries, soft flesh, neutral taste, and no flesh coloration, primarily used for winemaking.
5. Elliptic cross section, brownish color primocanes and floricanes with no lenticels and short-medium internodes (10-11 cm) of 10 mm diameter.
6. Harvesting time in early September (Middle Friuli, North-Eastern Italy); and
7. Resistance to winter temperature (−23° C.), resistance to downy mildew, and tolerance to powdery mildew.

Plants of the new grapevine ‘SAUVIGNON DORÉ’ differ from plants of the parents, Vitis vinifera ‘Sauvignon’ (unpatented) and ‘Bianca’ (unpatented), in the following characteristics described in Table 1.

TABLE 1
Comparison with Parent Varieties

| | New Cultivar | Female Parent | Male Parent |
|---|---|---|---|
| Charac- | ‘SAUVIGNON | ‘Sauvignon’ | ‘Bianca’ |
| teristic | DORÉ’ | (unpatented) | (unpatented) |
| vigor | medium | medium | strong |
| growth | Semi-erect | Semi-erect | semi-erect |
| habit | | | |
| leaf | medium size, | Medium size, | medium size, |
| | pale green col- | green color (up- | pale green color |
| | or (upper sur- | per surface), pale | (upper surface), |
| | face), pale green | green color (lower | green color (lower |
| | color (lower sur- | surface), circular | surface), no hairs |
| | face), wedge- | shape, medium | in both surfaces, |
| | shaped, medium | texture, prostrate | wedge-to-kidney- |
| | texture, no hairs | hairs present in | shaped, medium |
| | in both surfaces | upper surface and | texture |
| | | in lower surface | |
| cluster | Low size, cylin- | Low size, cylin- | Low size, conical, |
| | drical with 1-2 | drical-conical | with 1-2 wings, |
| | middle size | with 1-2 wings, | medium bunch |
| | wings, very | dense, berry skin | density, berry skin |
| | dense, berry skin | with green-yel- | with green-yellow |
| | with green-yellow | low color, slightly | color, slightly firm |
| | color, soft flesh, | firm flesh, herba- | flesh, neutral taste, |
| | neutral taste, no | ceous taste, no | no flesh coloration |
| | flesh coloration | flesh coloration | |
| primo- | elliptic cross sec- | Circular section, | oblate cross section, |
| cane and | tion, brownish | brownish color, | brownish color, |
| floricane | color, lenticels: | lenticels: absent, | lenticels: absent, |
| | absent, short- | short-middle | medium internodes, |
| | middle inter- | internodes (10- | diameter small |
| | nodes (10-11 | 11 cm), diameter | (about 8 mm) |
| | cm), diameter: | 10 mm | |
| | 10 mm | | |
| harvest- | Medium (early | Medium (Early | early (end of August |
| ing time | September in | September in | to mid-September |
| | Middle Friuli, | Middle Friuli, | in Middle Friuli, |
| | North-Eastern | North-Eastern | North-Eastern |
| | Italy) | Italy) | Italy) |
| resis- | resistant to win- | Average resis- | resistant to cold not |
| tances | ter temperature | tance to winter | known; resistant to |
| | (−23° C.), resis- | temperature (−15° | downy mildew, |
| | tant to downy mil- | C.), susceptible to | resistant to |
| | dew, tolerant to | downy mildew, | powdery mildew |
| | powdery mildew, | susceptible to | |
| | | powdery mildew | |

Of the many commercial cultivars known to the present inventor, the most similar to the new grapevine ‘SAUVIGNON DORÉ’ is the female parent ‘Sauvignon’, to which a comparison has been provided above.

### BRIEF DESCRIPTION OF THE PHOTOGRAPHS

The accompanying photographs illustrate the overall appearance of the new grapevine ‘SAUVIGNON DORÉ’ showing the colors as true as is reasonably possible with colored reproductions of this type. Colors in the photographs may differ slightly from the color values cited in the detailed botanical description, which accurately describe the color of ‘SAUVIGNON DORÉ’.

FIG. 1A and FIG. 1B show a typical fruit cluster of ‘SAUVIGNON DORÉ’, taken on Sep. 7, 2012 in Udine, Italy.

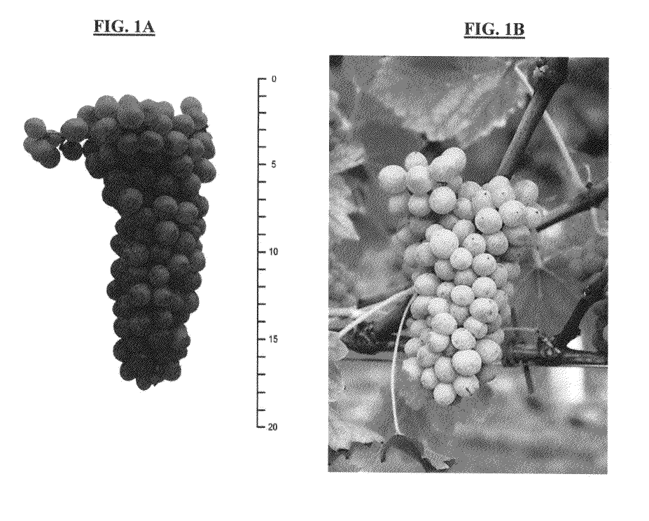

FIG. 2 shows a typical mature leaf (upper surface, left and lower surface, right) of ‘SAUVIGNON DORÉ’, taken on Jun. 15, 2012 in Udine, Italy.

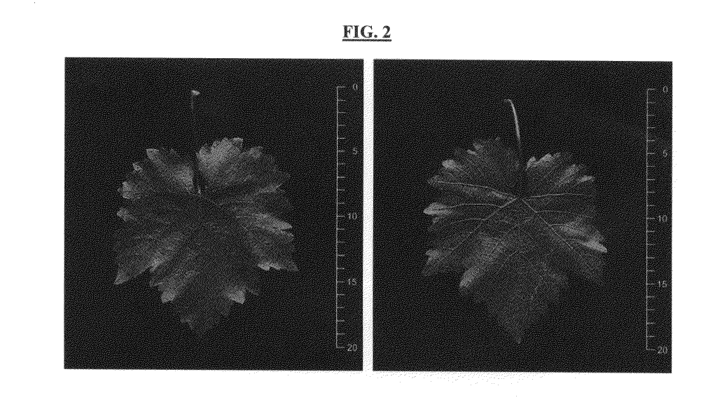

FIG. 3 shows a typical mature vine of ‘SAUVIGNON DORÉ’, taken on Aug. 3, 2012.

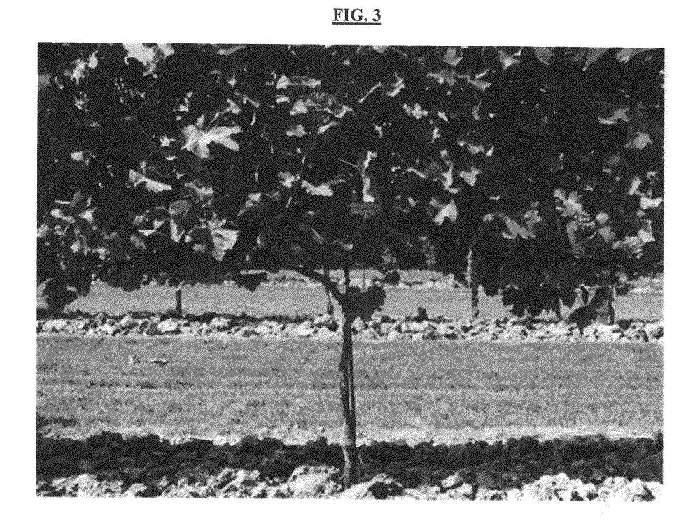

FIG. 4 shows a close-up view of typical mature fruit of ‘SAUVIGNON DORÉ’, taken on Aug. 25, 2012 in Udine, Italy

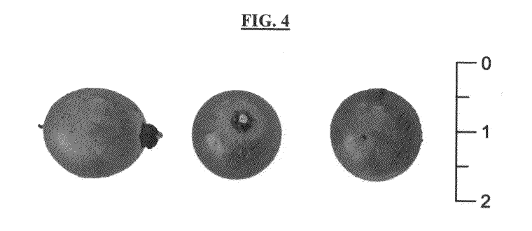

### DETAILED BOTANICAL DESCRIPTION

The following description of ‘SAUVIGNON DORÉ’ unless otherwise noted, is based on observations taken during the 2011, 2012, and 2013 growing seasons(s) in Udine (UD), Italy and Fossalon di Grado (GO), Italy, from plants dug from a nursery located in Rauscedo (PN), Italy during the beginning of December 2007 and planted approximately 16 to 20 weeks later in Udine (UD), Italy and Fossalon di Grado (GO), Italy. The phenotypical descriptions and color designations stated for the new variety may vary, depending upon variations in environmental factors, including weather (temperature, humidity and light intensity), day length, soil type, location and cultural conditions. ‘SAUVIGNON DORÉ’ has not been observed under all possible environmental conditions.

| | | |
|---|---|---|
| | ‘SAUVIGNON | ‘SAUVIGNON’ |
| Characteristic | DORÉ’ | (unpatented) |

GENERAL

| | | |
|---|---|---|
| Resistance to | Resistant to downy | Susceptible to downy |
| pest/disease | mildew, tolerant to | mildew and to pow- |
| | powdery mildew | dery mildew |
| Resistance to | Resistant to winter | Resistant to winter |
| natural elements | cold down to −23° C. | cold down to −15° C. |

VINE

| | | |
|---|---|---|
| Vigor | Medium | medium |
| Trunk diameter | 6 cm | 6.5 cm |
| Bark | | |
| color | Dark brown | Dark brown |
| underbark color | Light brown | Light brown |
| texture | n.a. | n.a. |
| Canes | | |
| length | Internode length | Internode length |
| | cm 10.5 | about cm 11 |
| width | 10 mm | 8 mm |
| Shoots | | |
| shape | Semi-erect, elliptic | Semi-erected, cross |
| | cross section, surface | section: circular, |
| | with stripes, nodes | surface: smooth, |
| | and internodes without | nodes and internodes |
| | hairs | without hairs |
| color | Internodes with red and | Internodes with green |
| | green pigmentation on | color on ventral and |
| | the dorsal side and | dorsal side, nodes with |
| | green on the ventral side | green ventral and dor- |
| | | sal side |
| Tendrils | | |
| form | Bifid | bifid |
| color | Green | Green |
| texture | normal | normal |
| number | 2 or <2 consecutive | 2 or <2 consecutive |
| Buds | | |
| size | average | average |
| shape | round | round |
| color | Light brown | Light brown |
| number | 2/node | 2/node |
| time of budbreak | medium | early-medium |

LEAVES

| | | |
|---|---|---|
| Size | medium | medium |
| Number of leaflets | 3-5 | 5 |
| Glossiness | Medium-high | medium |
| Cross section shape | V-shaped | V-shaped |
| Color (immature) | | |
| Upper surface | Pale green | Pale green |
| Under surface | green | Pale green |
| Color (mature) | | |
| Upper surface | Pale green | Medium green |
| Under surface | Pale green | Darker green |
| Petiole | | |
| Length (cm) | 6.2 | 6.0 |
| Color (upper | green | green |
| surface) | | |
| Color (under | Sometimes reddish | green |
| surface) | | |
| Stipule orientation | n.a. | n.a. |

FLOWERS

| | | |
|---|---|---|
| Flowering period | End of May | End of May |
| (time of beginning | | |
| of flowering) | | |
| Sex | hermaphrodite | hermaphrodite |
| Size | Average | average |
| Diameter (cm) | 0.3 (stamens) | 0.3 (stamens) |
| Fragrance | average | average |
| Flower number (at | n.a. | n.a. |
| 3^{rd} node from tip of | | |
| lateral mean and | | |
| range) | | |
| Petals | | |
| Length (cm) | n.a. | n.a. |
| Width (cm) | n.a. | n.a. |
| Overall shape | calyptra | calyptra |
| Color (immature) | green | green |
| Upper surface | | |
| Under surface | | |
| Color (mature) | green | green |
| Upper surface | | |
| Under surface | | |
| Sepals | | |
| Length (cm) | n.a. | n.a. |
| Width (cm) | n.a. | n.a. |
| Overall shape | n.a. | n.a. |
| Color (immature) | n.a. | n.a. |
| Upper surface | | |
| Under surface | | |
| Color (mature) | n.a. | n.a. |
| Upper surface | | |
| Under surface | | |
| Pedicel | | |
| Length (mm) | 7 | 7 |
| Color | green | green |

FRUIT

| | | |
|---|---|---|
| Primocane time of | September 17th | September 7th |
| fruiting (1^{st} pick) | | |
| Clusters | | |
| cluster weight | Low (g 192) | Low (g 230) |
| cluster shape | cylindrical | conical |
| cluster length | mm 180 | medium |
| avg. berries per | 150 | 220 |
| cluster | | |
| avg. clusters per | 2 | 2 |
| shoot | | |
| Berries | | |
| Berry size | g 1.3 | g 1.0 |
| Berry length (cm) | 1.68 | 1.50 |
| Berry width (cm) | 1.40 | 1.50 |
| Overall shape of | ellipsoid | globose |
| berry | | |
| Berry Texture | soft | soft |
| Berry Color | green | green |
| (immature) | | |
| Berry Color | Yellow-green | green |
| (mature) | | |
| Soluble solids (%) | 22.6 | 21.1 |
| Titratable acidity | 4.8 | 5.7 |
| (as g/L tartaric | | |
| acid) | | |
| Sugar/acid ratio | 4.7 | 3.7 |
| Firmness | soft | soft |
| Seeds | 2-3 | 2-3 |
| Skin cracking? | no | rarely |
| Juice color | transparent | transparent |
| Berry taste | Neutral | neutral |
| Eating quality | n.a. | n.a. |
| Berry uses | wine | wine |
| Shipping quality | n.a. | n.a. |

What is claimed is:

1. A new and distinct grapevine, referred to as ‘SAUVIGNON DORÉ’, as herein described and illustrated by the characteristics set forth above.
